(19) United States
(12) Patent Application Publication
(10) Pub. No.: US 2015/0005399 A1
(43) Pub. Date: Jan. 1, 2015

(54) METHOD AND DEVICE FOR PRODUCING SYNTHETIC GAS AND METHOD AND DEVICE FOR SYNTHESIZING LIQUID FUEL

(71) Applicants: Masayasu Sakai, Nagasaki-shi (JP); Nobuaki Murakami, Nagasaki-shi (JP); Nobutaka Morimitsu, Toyota-shi (JP); Yasunori Takei, Toyota-shi (JP); Akira Hasegawa, Isahaya-shi (JP)

(72) Inventors: Masayasu Sakai, Nagasaki-shi (JP); Nobuaki Murakami, Nagasaki-shi (JP); Nobutaka Morimitsu, Toyota-shi (JP); Yasunori Takei, Toyota-shi (JP); Akira Hasegawa, Isahaya-shi (JP)

(21) Appl. No.: 14/367,188

(22) PCT Filed: Nov. 29, 2012
(86) PCT No.: PCT/JP2012/080996

§ 371 Date: Jun. 19, 2014

(30) Foreign Application Priority Data
Dec. 20, 2011 (JP) 2011-279029

(51) Int. Cl. C01B 3/32; C07C 29/151; C07C 1/04

(52) U.S. Cl. CPC C01B 3/323; C07C 1/0485; C07C 1/041; C07C 29/1518; C01B 2203/0216; C01B 2203/1258; C01B 2203/0805
USPC 518/704; 422/129; 252/373; 422/162

(57) ABSTRACT

A method for producing synthetic gas with which a virtually soot-free synthetic gas having a good composition can be efficiently obtained by a simple device using a liquid biofuel as the starting material, and it is thereby possible to produce a high-quality liquid fuel such as methanol, gasoline or kerosene. Steam and a liquid biofuel produced by pyrolysis of a biomass are fed to the gasification space inside a reactor tube that is not loaded with a catalyst inside the reactor tube and heated to 800 to 1,200° C. from the outside via the reactor tube walls to induce an endothermic reaction and thereby a steam reforming chemical reaction between the steam and the liquid biofuel. By setting the molar ratio of the fed steam and carbon in the liquid biofuel ([H₂O]/[C]) at 0.3 or higher, a synthetic gas having a good composition that is virtually free of tar and soot and is primarily H₂ and CO is obtained.

### TECHNICAL FIELD

The present invention relates to effective utilization of biomass, and more specifically, to a method of generating, from biomass, a synthesis gas that serves as a high-quality and clean chemical raw material. Still more specifically, the present invention relates to a technology for enabling industrial production of a synthesis gas that contains high proportions of hydrogen H₂ and carbon monoxide CO and serves as a chemical raw material, which has hitherto not been able to be obtained from biomass.

### BACKGROUND ART

Biomass is generally a solid, and hence lacks convenience in combustibility, collection, transportation and the like, resulting in a problem in economic efficiency. Accordingly, recently, a liquid fuel generated from the biomass (also called biooil; hereinafter referred to as liquid biofuel) has been expected to be a practical fuel.

The liquid biofuel is obtained by using solid biomass such as grass or wood as a raw material, and converting the raw material into a liquid, which is easy to handle, through thermal treatment (such as fast pyrolysis). Specifically, the liquid biofuel can be produced from, for example, waste wood, a renewable product of agriculture and forestry such as grass or bark, or waste. In a general production method, first, such material is crushed and then rapidly heated under an oxygen-free state at from 400 to 500° C. to provide a liquid biofuel at a conversion rate of generally from 50 to 60% (weight ratio), and as by-products, a gas containing methane and the like as its components, soot containing carbon as a main component, and a solid carbonaceous (charcoal-like) substance are generated.

The liquid biofuel can be used as it is as a fuel. However, the liquid biofuel has a tar-like form with a high viscosity, and hence its use is limited to a low-quality fuel use such as one for burner combustion. The liquid biofuel is a hydrocarbon-based fuel, and hence has the potential to be processed into a more valuable fuel or chemical substance. At present, however, there is no technology for producing, from the liquid biofuel, a gas for synthesizing, for example, an automobile fuel.

In terms of chemistry, petroleum and refined products thereof such as gasoline, kerosene, light oil, and heavy oil are a mixture of hydrocarbons containing no oxygen, but the liquid biofuel contains a large amount of a compound having an oxygen atom in addition to carbon and hydrogen. Therefore, the liquid biofuel has a high degree of acidity and requires attention in, for example, selection of its container.

Meanwhile, research has been conducted to upgrade the liquid biofuel into a commercial product as a general liquid fuel. At present, however, this is successful only to the extent of providing a fuel of a low quality like inferior heavy oil.

### PRIOR ART LIST

### Patent Literature

[Patent Literature 1] JP 2009-001826 A

### SUMMARY OF INVENTION

### Technical Problem

From the viewpoints of the current global situation, the conservation of the natural environment, and the creation of a sustainable society, it has been becoming more and more important to develop an alternative resource to petroleum. However, among solar power, wind power, and biomass, which are regarded as representatives of sustainable energy, only the biomass can yield a renewable substance. In this sense, there is an urgent demand to develop a technology for producing a renewable substance including a fuel form originated from biomass.

The inventors of the present invention have previously proposed an external heating steam gasification method involving using solid plant biomass as a raw material (JP 2009-001826 A). This gasification method is a technology involving: in the absence of any catalyst, feeding finely pulverized biomass into a reaction tube filled with high-temperature steam and heating the reaction tube from outside to subject the steam and biomass to a steam reforming reaction in a furnace having a low oxygen concentration in the absence of any catalyst, to thereby generate a high-quality synthesis gas containing hydrogen and carbon monoxide as main components. The method can provide a good-quality synthesis gas directly from solid biomass.

However, the above-mentioned process involves directly gasifying solid biomass, and hence has the following problem(s): there is a difficulty in the handling of the raw material; and/or it is not easy to scale up a plant; and/or biomass, the ash of which has a low-melting point (for example, 800° C. or less), cannot be used. In view of this, the inventors of the present application have conceived a production method for a synthesis gas involving using a liquid biofuel, which is obtained through pyrolysis of biomass, as a raw material.

As a technique for obtaining a synthesis gas from hydrocarbon, there is known steam reforming. However, this method is only known to be used in the case of using, as a raw material, a gaseous or low-boiling point (generally 250° C. or less) hydrocarbon such as natural gas (main component; methane) or naphtha. In this method, the use of a catalyst is essential, and Ni is often used. The temperature of an outlet is kept from 800 to 950° C. during operation and a reforming furnace therefor has its inside lined with refractory bricks. A large number of cylindrical reaction tubes filled with an Ni catalyst are suspended in reforming furnace. The reforming furnace is heated from outside to supply heat needed for a reforming reaction to proceed. The heating is generally performed by combusting the same fuel as the raw material at a side wall. Optimal values for an operation pressure or the like is selected in consideration of factors including gas purification at a later stage, a target product, and the like, and is about 1-10 MPa.

This method is not applicable to a liquid heavier (having a higher boiling point) than naphtha, i.e., heavy oil or the like, and a partial oxidation method is used for such liquid. The Texaco process, the Shell process, and the like are well known partial oxidation methods. The partial oxidation method involves combusting part of a heavy oil raw material using oxygen obtained from air by a cryogenic separation method, to thereby provide high temperature needed for the reaction. The reaction temperature is from 1,300 to 1,500° C. The pressure is determined depending on gas purification at a later stage, a target product, and the like as in the case of hydrogen reforming and is from 1 to 8 MPa. This process involves operation at high temperature. Hence waste heat recovery and generation of soot are unavoidable and efforts are made into recovery/effective utilization thereof.

In contrast, hitherto, generation of a synthesis gas from a liquid biofuel has never been studied. The liquid biofuel has not been recognized as a material for steam reforming because of its properties (being like tar and having a high viscosity and a high boiling point) and the like. The application of the partial oxidation process is conceivable. In this case, however, part of the liquid biofuel is used in combustion, and hence it is conceivable that the amounts of effective H₂ and CO components in the generated synthesis gas decreases. In any case, partly because it is only recently that the liquid biofuel has started to attract attention, there is found no literature that makes a particular mention of the production of a high-quality liquid fuel such as gasoline, light oil, or methanol from the liquid biofuel.

### Solution to problem

The present application discloses the following inventions.

(1) A production method for a synthesis gas, comprising: supplying a steam and a liquid biofuel generated through pyrolysis of biomass to a gasification space in a reaction tube; and heating the gasification space from outside through a tube wall of the reaction tube to cause a steam reforming reaction to occur.
(2) A production method for a synthesis gas according to the above mentioned item (1), wherein the liquid biofuel is obtained by separating a liquid part from a product generated through pyrolysis of a solid biomass.
(3) A production method according to the above mentioned item (1) or (2), wherein the gasification space is free from a catalyst.
(4) A production method according to any one of the above mentioned items (1) to (3), wherein a molar ratio of the steam supplied to the gasification space to a carbon in the liquid biofuel is 0.3 or more.
(5) A production method according to any one of the above mentioned items (1) to (4), wherein the gasification space is heated to from 800° C. to 1,200° C.
(6) A production method according to any one of the above mentioned items (1) to (5), wherein a pressure in the gasification space is from 0.1 to 10 MPa.
(7) A production method according to any one of the above mentioned items (1) to (6), wherein: the liquid biofuel has a viscosity of from 10 to 50 centistokes; and the liquid biofuel is supplied to the gasification space by spraying.
(8) A production method according to any one of the above mentioned items (1) to (7), wherein the liquid biofuel is generated by heating a solid biomass to from 400 to 500° C. without actively performing deoxidation treatment.
(9) A production method according to any one of the above mentioned items (1) to (8), wherein the steam reforming reaction comprises a chemical reaction of the following formula [1], where q1=45 to 55%, q2=20 to 30%, q3=8 to 12%, and q4=15 to 25%, p1 is about 0.3 when a temperature in the gasification space is 800° C. and p1 is about 1.0 when the temperature in the gasification space is 1,000° C.

CₘH₂Oₙ+p1H₂O→q1H₂+q2CO+q3CH₄+q4CO₂ [1]

(10) A device for producing a synthesis gas, comprising: a reaction tube having a gasification space separated from an outside by a tube wall; a supply tube for supplying a steam and a liquid biofuel generated through pyrolysis of biomass to the reaction tube; and heating means for heating the gasification space from outside through the tube wall.
(11) A device and a method for synthesizing a liquid fuel configured to produce a hydrocarbon-based liquid fuel such as methanol, gasoline, or light oil through chemical synthesis using, as a raw material, a gas containing hydrogen and carbon monoxide as main components, the gas being obtained by the production method according to the above mentioned item (1).

The inventors of the present invention had a conviction that a synthesis gas can be generated through a reaction of a liquid biofuel using steam (steam reforming), because the liquid biofuel is an oxygen-containing fuel. Then, the inventors have found that the liquid biofuel can be converted into a high-quality synthesis gas without utilizing any catalyst and without needing a high temperature of from 1,300 to 1,500° C. to attain the invention according to the above-mentioned item (1), through experiments on various conditions including the addition amount of the steam.

In each of the inventions according the above-mentioned items (1) and (10), although it is necessary to first convert a solid into a liquid fuel, the following excellent effect(s) can be achieved: even biomass, the ash of which has a low-melting point of 800° C. or less and which cannot be used as a raw material in the prior process (JP 2009-001826 A), can be utilized irrespective of the melting point of the ash thereof; and/or the handling of the raw material is markedly facilitated; and/or the collection and transportation of the raw material become easy; and/or ash, foreign matter and the like are removed before supply to a gasification plant, and hence the plant can be built with a simple structure economically, thereby enabling development into a large-scale plant having high economic efficiency.

As compared to a process involving directly utilizing solid biomass, the process of the present invention involving two stages, i.e., liquefaction and gasification, may reduce total thermal efficiency in some cases. However, which of the processes is more economical depends on, for example, a distance between the location of the biomass raw material (solid) and the gasification plant. That is, it is assumed that: when the scale is so small that solid biomass can be locally produced and consumed, it is often advantageous in terms of thermal efficiency and plant cost to treat the biomass as a solid; and on the other hand, when the gasification plant has a large treatment capacity, the present invention, in which the biomass is first turned into a liquid at a few to several tens of biomass locations and then transported and collected to the gasification plant to be processed, is more excellent in economic efficiency in many cases.

It is preferable that the tube wall of the reaction tube is capable of providing heat needed for the steam reforming reaction from outside to the gasification space by radiation or the like; and separating the gasification space from an outside space (blocking an inflow and an outflow of substances (molecules and particles) between the gasification space and the outside space).

The molar ratio of the steam to be supplied (supplied steam) to the gasification space to carbon in the liquid biofuel ([H₂O]/[C]) is preferably 0.3 or more. With this, it is possible to: effectively prevent the generation of soot during the steam reforming reaction; and/or increase the amounts of hydrogen and CO in the synthesis gas. The above specified molar ratio is more preferably 0.5 or more, still more preferably 1 or more, particularly preferably 3 or more. The upper limit of the above specified molar ratio may be 30 or less. This is because the supply of the steam in larger amount than the above does not make a difference in soot-preventing effect. The above specified molar ratio is more preferably 20 or less, still more preferably 15 or less.

As the temperature in the gasification space (reaction temperature) becomes higher, the amounts of hydrogen and CO in the synthesis gas tend to increase. The temperature in the gasification space is preferably 800° C. or more, more preferably 850° C. or more, still more preferably 900° C. or more. Considering the heat resistance of the reaction tube, the temperature in the gasification space is preferably 1,200° C. or less, more preferably 1,150° C. or less, still more preferably 1,100° C. or less.

The invention according to the above-mentioned item (1) is also highly characterized in that the pressure in the gasification space can be 20 MPa or less during operation. The pressure in the gasification space is more preferably 15 MPa or less, still more preferably 10 MPa or less. The lower limit of the pressure in the gasification space is preferably 0.1 MPa or more, more preferably 0.3 MPa or more, still more preferably 0.5 MPa or more.

The chemical synthesis of methanol, ethanol, light oil, gasoline, or the like using the synthesis gas as a raw material is performed under a pressure as low as about from 5 to 10 MPa. Accordingly, when the pressure in the gasification space is from 5 to 10 MPa, the gasification of the liquid biofuel and the chemical synthesis of gasoline or the like can be easily performed in a continuous line.

Supply of the liquid biofuel to the gasification space is preferably performed by spraying. With this, an effect such as the facilitation of pressure control in the gasification space can be achieved. In order to facilitate the spraying, the liquid biofuel is preferably heated so as to have a viscosity of from 10 to 50 centistokes.

The liquid biofuel is preferably generated by: rapidly heating biomass to from 400 to 500° C.; and separating and removing a gas biofuel and solid residue char generated during the heating. In this case, by not actively performing deoxidation treatment, a liquid biofuel containing oxygen-containing liquid molecules as main components can be generated. The heating may be performed by electric or electromagnetic heating, fluidized-bed heating, kiln heating, or the like.

### BRIEF DESCRIPTION OF DRAWINGS

FIG. 1 is an explanatory diagram showing the composition of synthesis gases generated by the present invention.

FIG. 2 is an explanatory diagram illustrating an exemplary method and device for producing a synthesis gas according to the present invention.

### DESCRIPTION OF EMBODIMENTS

### Example 1

Used as a test device was a flow-type reaction device having provided therein a reaction tube made of SUS having an inner diameter of 54 mm and a length of 900 mm in an upright position, the reaction tube being electrically heated uniformly from its circumference. The space having an inner diameter of 54 mm and a length of 900 mm in the reaction tube serves as a gasification space. On the upstream side (below the gasification space), there was provided a steam-generating device of an electric furnace heating system, and a steam was supplied from the bottom portion of the reaction tube. In this upward flow, a sample is supplied with a microfeeder via an insertion tube having an inner diameter of 5 mm. On the downstream side after the reaction (above the gasification space), there are provided a filter for collecting soot in the synthesis gas that has exited the reaction tube, a condenser for cooling the generated gas, and a drainage bottle.

The gasification space is heated to a predetermined temperature, and nitrogen and a given amount of water are continuously flowed to the steam-generating device to generate the steam. Simultaneously with this upward flow, a given amount of the sample is supplied from the upper portion of the reaction tube. The sample is fed into the reaction tube, and a synthesis gas obtained by the reaction is released to the outside through the upper portion.

The synthesis gas was collected in a Tedlar bag, and its gas composition was analyzed using a gas chromatograph. In addition, the device was disassembled after the end of the test and the generation amounts of tar and soot were weighed. The gasification temperatures was set to either of 800° C., 900° C., and 1,000° C. The supply amount of the nitrogen as a carrier gas was set to 1 (L/min). A standard supply amount of the steam was set to 4 (g/min) and a standard supply amount of the sample was set to 1 (cc/min). The supply amount of the steam and the supply amount of the sample were changed as necessary. The reaction time in the standard condition was about 0.5 second. No catalyst was used, and the pressure was normal pressure.

It should be noted that the composition of the liquid biofuel used in this test is CH₂O_{0.53} when simply represented with using a carbon atom C as a reference, and has a property of an ordinary liquid biofuel. In addition, kerosene and heavy oil used for comparison are represented by CH_{2.67} and CH_{1.6}, respectively.

With the use of the above test device, first, an experiment for the liquid biofuel was performed to investigate the generation amount of soot at 900° C. with various molar ratios of the supplied steam to carbon in the fuel ([H₂O]/[C]). As a result, a soot generation rate (obtained by dividing the generation amount of soot by C in the supplied liquid biofuel; expressed on a mass basis) was 50% at the molar ratio of 0.2, and the test had to be stopped immediately. However, the soot generation rate lowered to: from 30 to 40% at the molar ratio of 0.3; 10% at the molar ratio of 0.5; and 4% at the molar ratio of 0.8, and was so small as to be hardly confirmed at the molar ratio of 1 or more. This tendency was similarly observed at 800° C., 1,000° C., and 1,100° C. The results revealed that it was important to maintain the molar ratio of the supplied steam to carbon in the fuel at 0.3 or more, preferably 0.5 or more in order to effectively perform gasification.

Next, a test regarding reactions of a liquid biofuel and petroleum-based fuels with a steam was performed. Table 1 shows a comparison of the results. In Table 1, the numbers of moles of a reaction water (H₂O that contributed to the reaction) and a generated product (C represents solid carbon) in the case where carbon C in each fuel is constant at 100 atom mol are shown (the shown value for H₂ is therefore doubled to yield the number of moles of a hydrogen atom). The generated gas and the effective gas are expressed in the numbers of moles of the gases and solid carbon ([C]) obtained in those conditions. CnHm represents a low molecular weight hydrocarbon (n=2, 3) in a gas form, and is shown as a value in terms of the number of moles of a carbon atom. The effective gas in the rightmost column refers to mol % of CO with respect to 100 of C when CO/H₂₌₂. All of the experiments were performed under a fixed condition in which; a molar ratio of the supplied steam to carbon in the fuel ([H₂O]/[C]) is 5.0 (H₂O/C weight ratio=4), a reaction temperature is 1,000° C., and a reaction time is about 0.5 second.

TABLE 1

| | | | Effective gas |
|---|---|---|---|
| Reaction system | | | CO mol |
| Raw material | Reaction water | Generated gas | % when |
| (molar ratio) | (molar ratio) | composition | CO/H₂ = 2 |

| | | | | | | | | |
|---|---|---|---|---|---|---|---|---|
| Liquid | H₂ | 100 | H₂ | 45.0 | H₂ | 114.9 | H₂ | 95.0 |
| biofuel | O | 53 | O | 45.0 | CO | 47.5 | CO | 47.5 |
| | C | 100 | | | CnCm | 15.5 | | |
| | | | | | CO₂ | 36.9 | | |
| | | | | | [C] | 0.2 | | |
| Kerosene | H₂ | 97.5 | H₂ | 20.0 | H₂ | 62.6 | H₂ | 23.0 |
| | O | 0.9 | O | 20.0 | CO | 11.5 | CO | 11.5 |
| | C | 100 | | | CnCm | 35.1 | | |
| | | | | | CO₂ | 4.2 | | |
| | | | | | [C] | 49.1 | | |
| Heavy oil | H₂ | 80.1 | H₂ | 11.8 | H₂ | 41.7 | H₂ | 9.4 |
| | O | 0.7 | O | 11.8 | CO | 4.7 | CO | 4.7 |
| | C | 100 | | | CnCm | 24.8 | | |
| | | | | | CO₂ | 3.5 | | |
| | | | | | [C] | 66.9 | | |

The following is found from Table 1.

(a) A great difference is found in synthesis gas composition between the liquid biofuel and the petroleum-based fuels. In addition, in the case of the petroleum-based raw materials, a filter outside the furnace was clogged in 15 minutes in the middle of the scheduled experiment time owing to a large amount of soot, and the experiment had to be stopped.
(b) The amount of soot discharged to the outside of the furnace is 1% or less of the raw material in the case of the liquid biofuel, while it is more than 40% in the case of the petroleum-based fuels. In addition, while the molar ratio of the supplied steam to the carbon in the fuel was 5, the molar ratio of the reaction water to the carbon in the fuel was about 0.45 as shown in Table 1.
(c) In the petroleum-based synthesis gas composition, the amounts of CO and CO₂ are extremely small, and the amounts of the generated solid carbon-based product and hydrocarbon gas are large.
(d) Taken together, the foregoing has revealed that, in a catalyst-free steam reforming reaction, as the raw material, a tar-like liquid biofuel allows a much larger amount of effective synthesis gas (H₂ and CO) to be obtained than petroleum-based ones, such as kerosene or crude oil, that have properties of being clear. This is presumably because an intramolecular oxygen bond in the liquid biofuel acts in gasification in a preferable way.

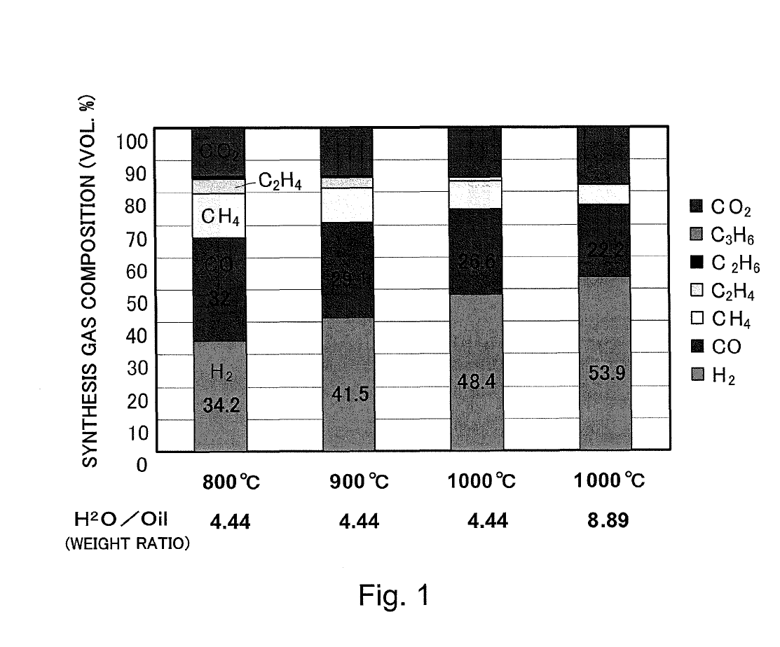

Next, experiments were performed under conditions allowing a larger proportion of hydrogen in the synthesis gas composition to be obtained and having a significant allowance for soot generation, namely a condition in which a molar ratio of [H₂O]/[C] was 5.5 (a weight ratio of H₂O/liquid biofuel=4.4) and a temperature in the gasification space was 800, 900, or 1,000° C., and a condition in which a molar ratio of [H₂O]/[C] was 11.1 (a weight ratio of H₂O/liquid biofuel=8.89) and a temperature in the gasification space was 1,000° C. In each temperature, satisfactory gasification with little carbon residue was achieved, and FIG. 1 shows the analysis results of the synthesis gas in each case. The results reveal that, in order to additionally increase the hydrogen/CO ratio, the conditions of a high temperature and a large H₂O supply amount are desirable.

Regarding a gas composition generated in the present gasification reaction, the followings are suggested from the obtained results and the like.

The formula [1] represents a target reaction in the present invention which can be caused to occur by setting a molar ratio of steam/biomass, calories of outside heating, and a reducing atmosphere (oxygen-deficient atmosphere) in the gasification space.

CₘH₂Oₙ+p1H₂O→q1H₂+q2CO+q3CH₄+q4CO₂ [1]

In the formula, CₘH₂Oₙ is a simple form of composition formula determined on the basis of elemental analysis. In general, m=1.2 to 1.6 and n=0.6 to 1.0. p1, which varies depending on the reaction temperature, is about 0.3 at 800° C. and about 1.0 at 1,000° C. q₁, q2, q3, and q4, which also vary depending on the reaction temperature, are as follows: q1=45 to 55%, q2=20 to 30%, q3=8 to 12% and q4=15 to 25%.

The synthesis gas composition varies depending not only on the reaction time, but also on the reaction temperature and the ratio of supplied steam/C molar as shown in the above mentioned FIG. 1 as an example. It can be understood that, in the gasification reaction of the present invention, in order to make H₂ and CO into a synthesis gas composition that can serve as a chemical raw material as it is, the reaction temperature is desirably from 900 to 1,000° C. It should be appreciated that it is possible to adopt a technique involving adjusting the ratio between CO and H₂ with a shift reactor at a later stage, or changing the gas composition with the gasification reaction temperature. In addition, although it can be assumed from the results that a higher temperature results in more satisfactory gas properties, a substantial upper limit is about 1,200° C. in view of the heat-resistant temperature of metal because of a feature of the present invention of heating the reaction tube from outside.

In addition, the quantity of heat that needs to be supplied from outside is found from thermal analysis of the steam reforming reaction to be from 30 to 54 kcal per 1 mol of carbon in the liquid biofuel.

It should be noted that the reaction pressure of the gasification may be desirably from 0.3 to 10 MPa when methanol synthesis is targeted. However, as in the case of a general chemical plant, the reaction pressure is specifically determined by considering all the factors including; the supply amount of H₂O, equipments at a later stage, that is, a shift reactor and a desulfurization reactor, synthesis conditions for target chemical substance (methanol, ethanol, FT synthetic oil such as light oil and gasoline, or DME), efficiency of auxiliary machines such as a compressor and costs. That is, the present invention is applied not to a solid raw material requiring a special supply device such as a lock hopper, but to a liquid raw material. Accordingly, a high-pressure supply device (such as a spraying device) can be easily used. In this case, when the gasification furnace is operated at a high operation pressure, though there arises a high cost factor such as the production of a pressurized vessel, there can be obtained advantages in, for example, that: the reaction tube can be reduced in size; and compression power needed for the pressurization of the synthesis gas can be saved.

### Example 2

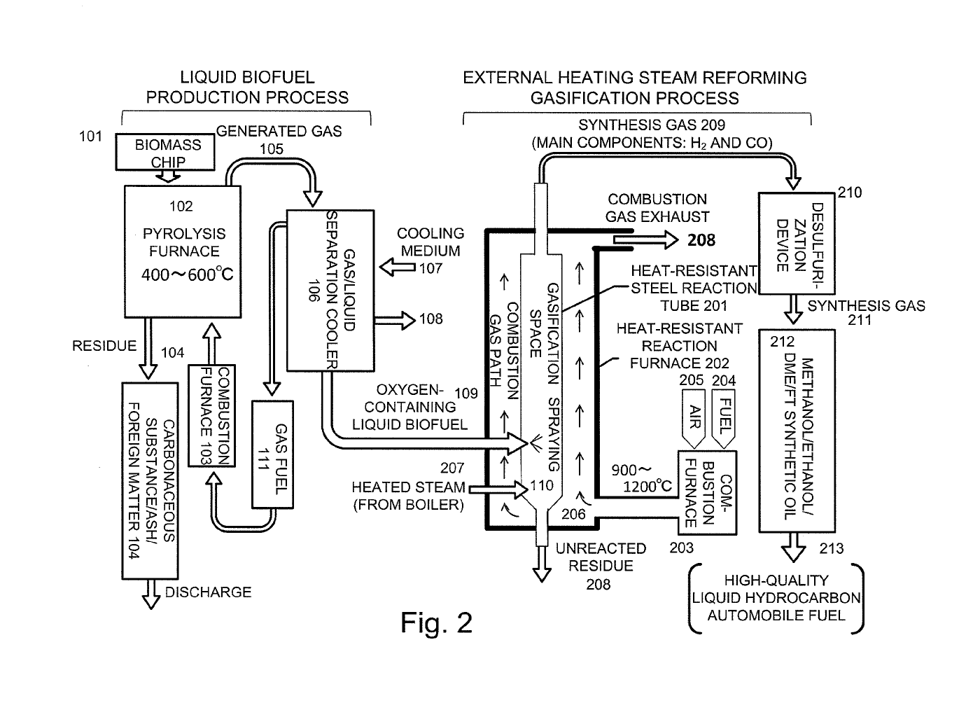

FIG. 2 is an explanatory diagram illustrating an exemplary method and device for producing a synthesis gas according to the present invention.

In the figure, a biomass chip 101 is heated to from 400 to 600° C. in a pyrolysis furnace 102 by a hot gas from a combustion furnace 103, and a solid residue (carbonaceous substance/ash/foreign matter) 104 generated during the heating is discharged from the bottom of the combustion furnace. A generated gas 105 taken out from the upper portion of the pyrolysis furnace 102 is separated into a gas fuel 111 and a liquid biofuel 109 in a gas/liquid separation cooler 106. Reference numerals 107 and 108 denote an inlet and outlet for a cooling medium, respectively.

In this state, generally, the liquid biofuel 109 takes an oxygen-containing hydrocarbon structure. This liquid biofuel is introduced to a heat-resistant reaction furnace 202. In the steam reforming gasification process of the present invention, the oxygen-containing liquid biofuel 109 is sprayed and atomized by a spraying nozzle 110 to be supplied to a gasification space in a reaction tube 201, and a steam 207 as a gasifying agent from the lower portion of the reaction tube and a reaction heat supplied by radiation heat from the reaction tube 201 cause a steam reforming reaction to occur. It should be noted that the spraying of the liquid biofuel is desirably performed by pressure spraying or steam spraying, and the liquid biofuel is preferably heated so as to have a viscosity of from 10 to 50 centistokes.

At this time, the reaction tube 201 is heated in the reaction furnace 202 from outside of the reaction tube 201 with a high-temperature combustion gas 206 at from 900 to 1,200° C. generated by combusting a fuel 204 and an air for combustion 205 in a combustion furnace 203. By this, the gasification space in the reaction tube 201 is heated to from 800° C. to 1,200° C. A liquid biofuel, its raw material, i.e., biomass, or the like is appropriately selected as the fuel therefor. Reference numeral 208 denotes an exhaust port for a combustion gas.

A synthesis gas 209, which is obtained by gasification through the steam reforming reaction in the reaction tube 201, can be utilized as a synthesis gas containing hydrogen H₂ and carbon monoxide CO as main components. It should be noted that, generally, sulfur content such as H₂S is removed with a desulfurization device 210 in order to prevent deterioration of a catalyst for chemical synthesis at a later stage. A purified synthesis gas 211 is used in a synthesis reactor 212 to produce a liquid fuel 213 such as methanol, ethanol, DME, gasoline, or light oil through the use of respective technique and catalyst.

By using the method of the present invention, a synthesis gas that hardly contains soot and has satisfactory composition can be obtained efficiently with a simple device using a liquid biofuel as a raw material. This enables the production of a high-quality liquid fuel such as gasoline, light oil and methanol. Under the current situation where utilization of biomass is being investigated throughout the world, the present invention is extremely useful in industry as a countermeasure against global warming and fossil fuel depletion.

### REFERENCE SIGNS LIST

101 biomass chip
102 pyrolysis furnace
103 combustion furnace
104 solid residue
105 generated gas
106 gas/liquid separation cooler
107 inlet for cooling medium
108 outlet for cooling medium
109 liquid biofuel
110 spraying nozzle
111 gas fuel
201 reaction tube
202 heat-resistant reaction furnace
203 combustion furnace
204 fuel
205 air for combustion
206 high-temperature combustion gas
207 exhaust port for combustion gas
208 exhaust port for combustion gas
209 synthesis gas
210 desulfurization device
211 purified synthesis gas
212 synthesis reactor

1. A production method for a synthesis gas, comprising:
supplying a steam and a liquid biofuel generated through pyrolysis of biomass to a gasification space in a reaction tube; and
heating the gasification space from outside through a tube wall of the reaction tube to cause a steam reforming reaction to occur.

2. A production method for a synthesis gas according to claim 1, wherein the liquid biofuel is obtained by separating a liquid part from a product generated through pyrolysis of a solid biomass.

3. A production method according to claim 1, wherein the gasification space is free from a catalyst.

4. A production method according to claim 1, wherein a molar ratio of the steam supplied to the gasification space to a carbon in the liquid biofuel is 0.3 or more.

5. A production method according to claim 1, wherein the gasification space is heated to from 800° C. to 1,200° C.

6. A production method according to claim 1, wherein a pressure in the gasification space is from 0.1 to 10 MPa.

7. A production method according to claim 1, wherein:
the liquid biofuel has a viscosity of from 10 to 50 centistokes; and
the liquid biofuel is supplied to the gasification space by spraying.

8. A production method according to claim 1, wherein the liquid biofuel is generated by heating a solid biomass to from 400 to 500° C. without actively performing deoxidation treatment.

9. A production method according to claim 1, wherein the steam reforming reaction comprises a chemical reaction of the following formula [1], where q1=45 to 55%, q2=20 to 30%, q3=8 to 12%, and q4=15 to 25%, p1 is about 0.3 when a temperature in the gasification space is 800° C. and p1 is about 1.0 when the temperature in the gasification space is 1,000° C.
CₘH₂O+p1H₂O→q1H₂+q2CO+q3CH₄+q4CO₂ [1]

10. A device for producing a synthesis gas, comprising:
a reaction tube having a gasification space separated from an outside by a tube wall;
a supply tube for supplying a steam and a liquid biofuel generated through pyrolysis of biomass to the reaction tube; and
heating means for heating the gasification space from outside through the tube wall.

11. A device for synthesizing a liquid fuel configured to produce a hydrocarbon-based liquid fuel such as methanol, gasoline, or light oil through chemical synthesis using, as a raw material, a gas containing hydrogen and carbon monoxide as main components, the gas being obtained by the production method according to claim 1.

12. A method for synthesizing a liquid fuel, comprising producing a hydrocarbon-based liquid fuel such as methanol, gasoline, or light oil through chemical synthesis using, as a raw material, a gas containing hydrogen and carbon monoxide as main components, the gas being obtained by the production method according to claim 1.
